(19) United States
(12) Patent Application Publication
(10) Pub. No.: US 2017/0299536 A1
(43) Pub. Date: Oct. 19, 2017

(54) GAS ANALYZER AND GAS ANALYSIS METHOD

(71) Applicant: FUJITSU LIMITED, Kawasaki-shi (JP)

(72) Inventors: Osamu Tsuboi, Kawasaki (JP); Satoru Momose, Atsugi (JP); Michio USHIGOME, Atsugi (JP); Kazuaki Karasawa, Hadano (JP); Ryozo Takasu, Isehara (JP)

(73) Assignee: FUJITSU LIMITED, Kawasaki-shi (JP)

(21) Appl. No.: 15/466,384

(22) Filed: Mar. 22, 2017

(30) Foreign Application Priority Data
Apr. 14, 2016 (JP) 2016-081274

(51) Int. Cl. G01N 27/12; G01N 33/497

(52) U.S. Cl. CPC G01N 27/125; G01N 27/123; G01N 33/497

(57) ABSTRACT

A gas analyzer including: a chamber; a first gas sensor provided in the chamber and including a first gas sensitive member; a second gas sensor provided in the chamber and including a second gas sensitive member; and a detector that detects each of resistance changes of the first and the second gas sensitive members; wherein the first gas sensitive member is an oxide semiconductor mainly composed of at least one of Sn, W, Zn and In or a semiconductor mainly composed of C, and the second gas sensitive member is mainly composed of a halide or an oxide of Cu or Ag.

### CROSS-REFERENCE TO RELATED APPLICATION

This application is based upon and claims the benefit of priority of the prior Japanese Patent Application No. 2016-081274, filed on Apr. 14, 2016, the entire contents of which are incorporated herein by reference.

### FIELD

A certain aspect of embodiments described herein relates to a gas analyzer and a gas analysis method.

### BACKGROUND

Each of Patent Documents 1-3 discloses a technique that detects each component from a gas containing a plurality of components (see e.g. Patent Document 1: Japanese Laid-open Patent Publication No. 03-163343, Patent Document 2: Japanese Laid-open Patent Publication No. 2008-292344, and Patent Document 3: Japanese Laid-open Patent Publication No. 2000-55853).

### SUMMARY

According to an aspect of the present invention, there is provided a gas analyzer including: a chamber; a first gas sensor provided in the chamber and including a first gas sensitive member; a second gas sensor provided in the chamber and including a second gas sensitive member; and a detector that detects each of resistance changes of the first and the second gas sensitive members; wherein the first gas sensitive member is an oxide semiconductor mainly composed of at least one of Sn, W, Zn and In or a semiconductor mainly composed of C, and the second gas sensitive member is mainly composed of a halide or an oxide of Cu or Ag.

The object and advantages of the invention will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating gas components in a breath;

FIG. 2 is a schematic diagram illustrating the whole configuration of a gas analyzer;

FIG. 3A is a diagram illustrating the whole configuration of gas sensors;

FIG. 3B is a top view of a substrate;

FIG. 3C is a bottom view of the substrate;

FIG. 4 is a diagram illustrating resistance change rates of CuBr, SnO₂ and WO₃ to gas components;

FIG. 5 is a diagram illustrating a result of a principal component analysis;

FIGS. 6A to 6C are diagrams illustrating a breathprint sensor;

FIG. 7 is a diagram illustrating a result of the principal component analysis;

FIG. 8 is a diagram illustrating a result of the principal component analysis; and

FIG. 9 is a diagram illustrating a result of the principal component analysis;

### DESCRIPTION OF EMBODIMENTS

In the above-mentioned technique of the Patent Documents 1-3, an excellent selection ratio cannot be obtained with respect to a gas containing both of a reducing gas and a basic gas.

First, a description will be given of an outline of a gas analysis. In the following embodiments, a gas to be measured contains both of the reducing gas and the basic gas, and is a biological gas (a breath, body odor, urine, fart, a stool) discharged from a body and an excrement of a human, an animal and so on, for example. A gas analyzer and a gas analysis method according to the present embodiment are used for medical care and health care, and specify each component of the biological gas, for example.

In an aging society accelerating more and more, the total sum of the medical expenses of the nation has a tendency to increase year by year. According to the statistics of Ministry of Health, Labour and Welfare of 2015, the total sum of the medical expenses of the nation exceeded 40 trillion yens in 2013, which became a social problem. With respect to the types of diseases, a ratio of diseases due to a lifestyle such as high blood pressure, diabetes and cancer occupies high ranks. For this reason, the need of early detection of a lifestyle-related disease increases. In such a background, a breath analysis for inspecting an index of a body condition from the biological gas and a study of a diagnosis method using the breath analysis are performed.

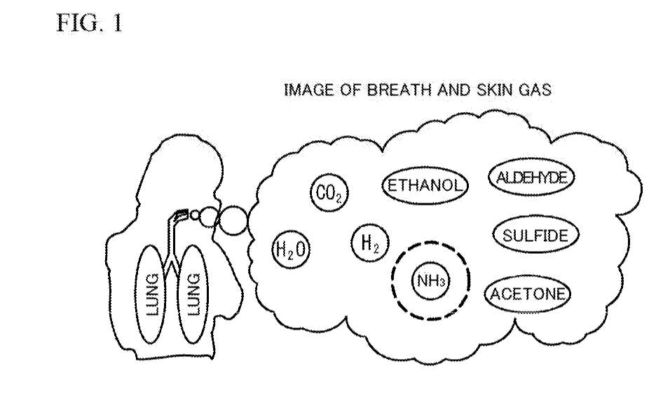

The extremely low-concentration gases are formed in the lungs by vaporizing a chemical substance in blood, and the extremely low-concentration gases discharged from the lungs are contained in a breath of the human and the animal, as illustrated in FIG. 1. The extremely low-concentration gases have components that are closely related to a biological activity and the disease. For example, it is said that an ammonia gas included in the breath of the human is correlated with the metabolism of a liver and helicobacter pylori infection that is a risk factor of a stomach cancer. Moreover, nonanal which is aldehydes is considered to be a candidate of a lung cancer marker material.

By analyzing the gases, the breath analysis aims to detect a specific substance effective for screening for the improvement of the lifestyle and the early detection of the disease with the use of a simple means that only blows a breath without the restriction of the body and a pain of blood collection.

However, a great many kinds of volatile gases (according to one theory, 200 kinds or more) are contained in the biological gas. Most of the biological gas are the reducing gas such as organic molecules (hydrocarbon), and chemical properties thereof are similar to each other. As methods of analyzing components of such a gas, there are roughly two types of methods.

In one method, by using a large-scale analyzer represented by a gas chromatography, a specific gas component is measured. In this method, the component of the gas can be analyzed in detail. However, the operation of an expert is required, it takes several hours or more until a result is obtained, and the analyzer is an expensive and large-scale device. Therefore, in this method, a burden of the inspection is large, so that this method is mainly used for research purposes.

In another method, by using an apparatus equipped with a large number of gas sensors, a difference between response patterns of the gas sensors depending on the gases is analyzed. In this method, the time required for acquiring the result of the analysis is short, and the apparatus is portable and can be easily used. On the other hand, a sensitivity difference between the sensors is small, and it is therefore difficult to distinguish the specific gas and other gases. Accordingly, this method is not sufficient for the breath analysis that inspects the index of the body condition.

In most of conventional gas sensors, a tin oxide is used as a base of the material. Gas molecules and oxygen are heated with a heater, and an adsorption amount of active oxygen to a gas sensitive member is detected as a resistance change of a semiconductor material, so that each component can be specified. A selectivity (i.e., a difference between strengths of the response according to the gas components) can be achieved by selection of a kind of a metal which is a main body, containing a noble metal with a gas catalytic action, a heating amount of the heater, and so on. However, at any rate, a balance of a reducibility and oxygen is only measured, and a selection ratio is not large. For example, the selection ratio is slightly smaller than 10, and there is not almost an orthogonality of characteristic vectors.

For example, it is considered to use the technique using gas sensor arrays of the above Patent Documents 1-3. However, the gas components can be classified by statistical processing, but a principal component direction moves for each population. Therefore, it is difficult to get a concentration index of the specific gas. That is, a reproducibility is worse and there is no quantitativity. On the contrary, in a system without using the gas sensor arrays such as an optical system or a vibration system, unit systems of the measurement results differ from each other according to a difference in a principle between a system using the gas sensor arrays and the system without using the gas sensor arrays, and hence data conversion is required. Therefore, it is difficult to perform the statistics processing with the quantitativity. Moreover, according to the difference in the principle of the above systems, measurement data are huge amount and cannot be combined.

Accordingly, in the following embodiments, a description will be given of a gas analyzer and a gas analysis method that can obtain an excellent selection ratio with respect to a gas containing both of the reducing gas and the basic gas with a simple configuration.

### EMBODIMENT

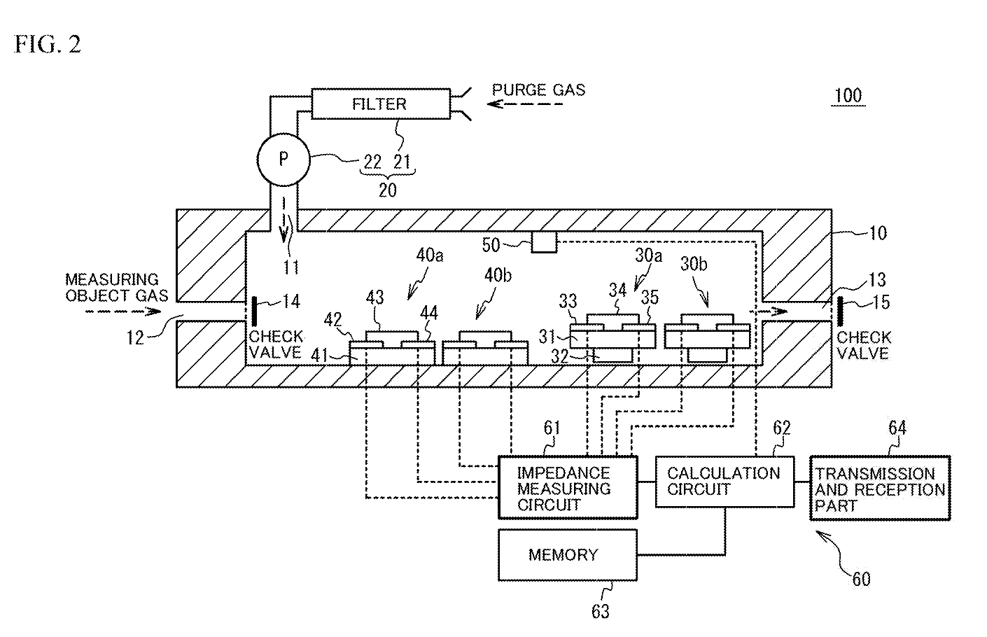

FIG. 2 is a schematic diagram illustrating the whole configuration of a gas analyzer 100. A gas which is a measurement object of the gas analyzer 100 contains both of the reducing gas and the basic gas. The reducing gas is a gas which is easily oxidized by oxygen, and is a hydrogen sulfide, or organic compounds such as alcohols, ketones, or the like. The reducing gas occurs in a process where a living body decomposes a hydrocarbon in particular. The basic gas is a gas having basicity, and is ammonia generated in a process where the living body decomposes a protein in particular.

As illustrated in FIG. 2, the gas analyzer 100 includes a purge gas supplier 20 on the outside of a chamber 10. Moreover, the gas analyzer 100 includes gas sensors 30a and 30b, gas sensors 40a and 40b, and a temperature and humidity sensor 50 on the inside of the chamber 10. The gas analyzer 100 includes a calculation unit 60 on the outside of the chamber 10. The calculation unit 60 includes an impedance measuring circuit 61, a calculation circuit 62, a memory 63, a transmission and reception part 64, and so on. The impedance measuring circuit 61 and the calculation circuit 62 serve as an example of a detector that detects the resistance change of the gas sensitive member mentioned later.

A first inlet 11, a second inlet 12 and an outlet 13 are formed on the chamber 10. The first inlet 11 is an opening for supplying a purge gas into the chamber 10. The second inlet 12 is an opening for supplying a measuring object gas into the chamber 10. The outlet 13 is an opening for discharging the purge gas or the measuring object gas from the chamber 10.

A pipe from the purge gas supplier 20 is connected to the first inlet 11. A check valve 14 is provided on the second inlet 12. The check valve 14 is configured to suppress gas outflow from the chamber 10 through the second inlet 12. A check valve 15 is provided on the outlet 13. The check valve 15 is configured to suppress gas inflow from the outside of the chamber 10 through the outlet 13.

The purge gas supplier 20 includes a filter 21 and a blast pump 22. The purge gas is not limited in particular, but is an air. The blast pump 22 sucks the purge gas through the filter 21 and supplies the purge gas into the chamber 10 through the pipe. The filter 21 removes dusts in the purge gas. The purge gas is supplied into the chamber 10 with the blast pump 22, so that an internal pressure of the chamber 10 rises and a gas inflow from the second inlet 12 is suppressed by an action of the check valve 14. When the internal pressure of the chamber 10 rises, the check valve 15 does not operate and hence the purge gas is discharged from the outlet 13. Thereby, the inside of the chamber 10 can be purged.

When the measurement of the gas is performed, the measuring object gas flows into the chamber 10 from the second inlet 12. The outflow of the measuring object gas from the second inlet 12 is suppressed with the check valve 14. The measuring object gas flows in the chamber 10, and is discharged from the outlet 13.

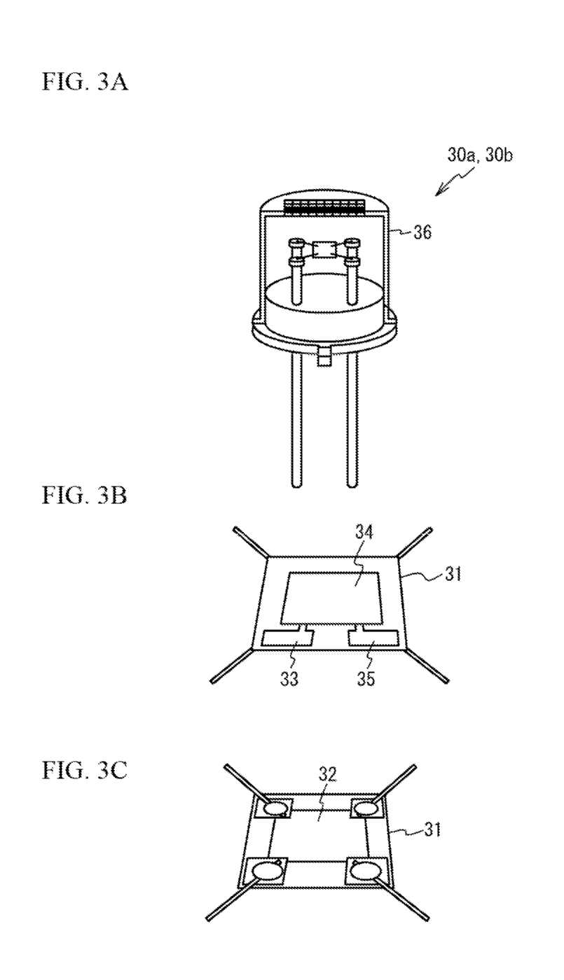

In each of the gas sensors 30a and 30b, a heater 32 is provided on a bottom surface of a substrate 31, and an electrode 33, a gas sensitive member 34 and an electrode 35 are provided on a top surface of the substrate 31. FIG. 3A is a diagram illustrating the whole configuration of the gas sensors 30a and 30b. The substrate 31, the heater 32, the electrode 33, the gas sensitive member 34 and the electrode 35 illustrated in FIG. 2 are arranged in a housing 36 having an opening in a part thereof. FIG. 3B is a top view of the substrate 31. FIG. 3C is a bottom view of the substrate 31. The substrate 31 is composed of an insulating material such as an alumina. The heater 32 is composed of a material generating a heat by electric supply, and is a NiCr thin film or the like. The electrode 33 is provided on one end of the gas sensitive member 34. The electrode 35 is provided on the other end of the gas sensitive member 34. Each of the electrodes 33 and 35 is connected to a terminal on the bottom surface of the substrate 31 through a via. Thereby, the heater 32 and the gas sensitive member 34 are connected in parallel.

The gas sensitive member 34 is composed of a material having high sensitivity with respect to a reducing gas concentration. The gas sensitive member 34 is an oxide semiconductor containing at least one of Sn (tin), W (tungsten), Zn (zinc) and In (indium) or a semiconductor mainly composed of C (carbon). When the gas molecules and oxygen in the housing 36 are heated with the heater 32, the adsorption amount of the active oxygen to the gas sensitive member 34 changes. The change of the adsorption amount of the active oxygen causes the change of the resistance of the gas sensitive member 34. By detecting the change of the resistance, the gas concentration to be measured can be detected.

By changing the kind of the metal constituting the oxide semiconductor, the selectivity (i.e., the difference between strengths of the response according to the gas components) can be given to the gas sensitive member 34. Alternatively, by containing the noble metal with the gas catalytic action into the gas sensitive member 34 or by changing the kind of the noble metal, the selectivity can be given to the gas sensitive member 34. For example, by containing an additive metal, such as Pd (palladium) or Pt (platinum) of the noble metal or Al (aluminum) or Pb (lead) of a base metal, into the gas sensitive member 34, the selection ratio between the kinds of gases can be decided. Alternatively, by changing the heating amount of the heater 32, the selectivity can be given to the gas sensitive member 34. Here, it is preferable to increase the sensitivity than around 0.1-10 times with respect to acetone, ethanol or the like.

To provide a sensitivity difference with respect to VOC (volatile organic compounds), an organic thin film may be formed on the gas sensitive member 34. Since the sensitivity of the gas sensitive member 34 relatively decreases when the organic thin film is formed, it is desirable to form the organic thin film as thin as possible. For example, by applying gold particles to the surface of the gas sensitive member 34 and exposing it to a high molecular gas, a monolayer may be formed. For example, it is preferable to use a coupling material of amine system, thiol system, silane system or the like.

Each of the gas sensors 30a and 30b can detect the gas component and the gas concentration by detecting the change of a resistance value of the gas sensitive member 34. In the gas sensors 30a and 30b, an optimum detection temperature exists depending on the kind of the gas to be detected. Therefore, at the time of the gas concentration measurement performing the gas detection, the inside of the housing 36 is set to the optimum detection temperature. The inside of the housing 36 is heated at a detection temperature in a detection temperature range including the optimum detection temperature by the heater 32. On the contrary, at the time of cleaning of the gas sensitive member 34, a temperature in the housing 36 is raised to a cleaning temperature higher than the temperature at the time of the gas detection, which make it possible to desorb contaminants absorbed to the surface of the gas sensitive member 34.

In each of the gas sensors 40a and 40b, an electrode 42, a gas sensitive member 43 and an electrode 44 are provided on the top surface of a substrate 41. The electrode 42 is provide on one end of the gas sensitive member 43. The electrode 44 is provided on the other end of the gas sensitive member 43. The gas sensitive member 43 is composed of a material having high sensitivity with respect to a basic gas concentration. Copper ions (Cu⁺ and Cu²⁺) and a silver ion (Ag⁺) exhibit a high affinity to the basic gas by existing as mobile ions. Therefore, in the present embodiment, the gas sensitive member 43 is mainly composed of a halide or an oxide of copper or silver. Copper bromide (I) (CuBr) which is a p-type semiconductor can be used as an example.

Since the copper ions and the silver ion have the high affinity to the basic gas, the basic gas is strongly adsorbed to the gas sensitive member 43. In this case, by detecting the change of the resistance of the gas sensitive member 43, the component and the concentration of the basic gas can be measured. For example, the copper ions and the silver ion form coordinate bond together with a nitrogen atom of amine. Thereby, the copper ions and the silver ion have the high affinity with nitrogen. Therefore, the basic gas such as ammonia can be measured by using the copper ions and the silver ion. An univalent copper ion has the high affinity with nitrogen, compared with a divalent copper ion. Therefore, it is preferable to use the halide or the oxide of the univalent copper ion.

Each of the gas sensors 40a and 40b uses the adsorption of the gas molecules, and hence the heating with the heater is not necessarily required. However, by lowering the temperature at the time of the adsorption and by raising the temperature at the time of the desorption, the sensitivity and the responsiveness can be improved.

To provide the sensitivity difference with respect to VOC (volatile organic compounds), the organic thin film may be formed on the gas sensitive member 43. Since the sensitivity of the gas sensitive member 43 relatively decreases when the organic thin film is formed, it is desirable to form the organic thin film as thin as possible. For example, by applying gold particles to the surface of the gas sensitive member 43 and exposing it to the high molecular gas, the monolayer may be formed. For example, it is preferable to use the coupling material of amine system, thiol system, silane system or the like.

The heat of the heater 32 might reach a downstream side along the flow of the gas. Therefore, it is preferable that the gas sensors 30a and 30b are arranged closer to the outlet 13 than the gas sensors 40a and 40b. Thereby, it is possible to suppress an influence of the heat of the heater 32 against the gas sensors 40a and 40b.

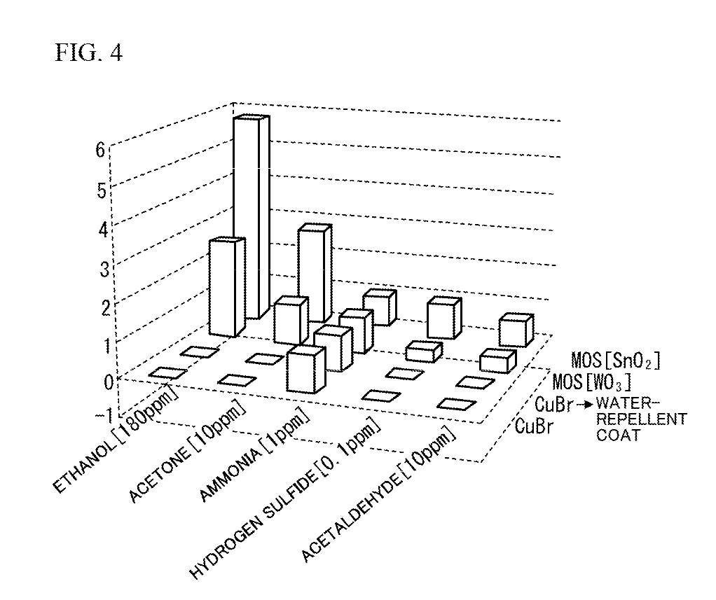

FIG. 4 is a diagram illustrating resistance change rates of CuBr, SnO₂ and WO₃ to the gas components. A vertical axis indicates a normalized value of the resistance change rate. Each of MOSs in FIG. 4 indicates a metal oxide semiconductor. With respect to CuBr, an uncoating CuBr and CuBr to which a water-repellent coat is applied are illustrated. The resistance change rate is normalized on the basis of a resistance change rate with respect to ammonia 1 ppm. As illustrated in FIG. 4, CuBr acquires an extremely large resistance change rate with respect to the basic gas (ammonia), compared with other gas components. On the other hand, in CuBr, the resistance hardly changes with respect to the reducing gas. This is because the halide or the oxide of copper or silver has a low affinity to the reducing gas. Therefore, the concentration of the basic gas can be accurately measured by using the gas sensor including CuBr as the gas sensitive member 43.

SnO₂ and WO₃ acquire large resistance change rates with respect to a plurality of gas components. However, the resistance change rates differ from each other according to the gas components. The concentration of each of the gas components can be accurately measured based on this difference. SnO₂ and WO₃ are different from each other in the resistance change rate with respect to each gas component. Therefore, the concentration of each of the gas components can be measured more accurately by using both of SnO₂ and WO₃.

Referring to FIG. 2 again, each electrode of the gas sensors 30a and 30b and the gas sensors 40a and 40b are connected to the impedance measuring circuit 61. Thereby, the impedance measuring circuit 61 measures the resistance of each of the gas sensitive members. Specifically, the impedance measuring circuit 61 measures an impedance of the gas sensitive member 34 of each of the gas sensors 30a and 30b, and measures an impedance of the gas sensitive member 43 of each of the gas sensors 40a and 40b. The impedance measuring circuit 61 transmits the result of the measurement to the calculation circuit 62. The calculation circuit 62 calculates a concentration of each gas component of the measuring object gas. The transmission and reception part 64 transmits a result of the calculation of the calculation circuit 62 to an external device.

Next, a description will be given of the calculation of the concentration of each gas component in the measuring object gas. In the present embodiment, a gas containing the reducing gas, the basic gas and a plurality of components is a measuring object. Each of the gas sensors does not have sensitivity only to a specific component. That is, even when the resistance change of the gas sensitive member of each of the gas sensors is measured, it is hard to specify which component the resistance change corresponds to. For this reason, in the present embodiment, a characteristic vector about a response to each gas component of each gas sensor is calculated beforehand by performing statistics processing or machine learning for a measurement result of the impedance of the gas sensitive member of each gas sensor. The calculated characteristic vector is stored into the memory 63.

First, a learning result of a response (an impedance change) of the gas sensors 30a, 30b, 40a and 40b with respect to a gas having a well-known component and a well-known concentration is stored beforehand into the memory 63 as matrix data. For example, a set (z11, z12 . . . , z1n) of the impedance change of the gas sensor 30a and a set (z21, z22 . . . , z2n) of the impedance change of the gas sensor 30b are set as element data. Similarly, with respect to the gas sensors 40a and 40b, the sets of the impedance change are stored into the memory 63. Each element of the sets of the impedance change is an impedance change amount, with respect to each of a plurality of gases having different components and different concentrations, after the gas is introduced and different time elapses. Alternatively, a correlation of the gas concentration and the impedance change amount with respect to a standard gas (e.g., ammonia) may be examined, and data converted into the concentration by using the correlation may be set as the element data.

Next, the calculation circuit 62 performs the statistics processing or the machine learning for the sets of the impedance change of the gas sensors 30a, 30b, 40a and 40b to calculate the characteristic vector of each of the gas sensors. In the present embodiment, the calculation circuit 62 calculates the characteristic vector of a first main axis, a second main axis and a third main axis as an example. Any one of a principal component analysis method, a multiple regression analysis method, a PCR (principal component regression) method, a SVD (singular value decomposition) method, a PLS (partial least squares) method, a LWR (locally weighted regression) method, a discriminant analysis method, a multi-layer perceptron method, a neural network, a SVM (support vector machine) method, and a leave-one-out method, or a combination of two or more kinds thereof can be used as the statistics processing or the machine learning. In the present embodiment, the principal component analysis method is used as an example.

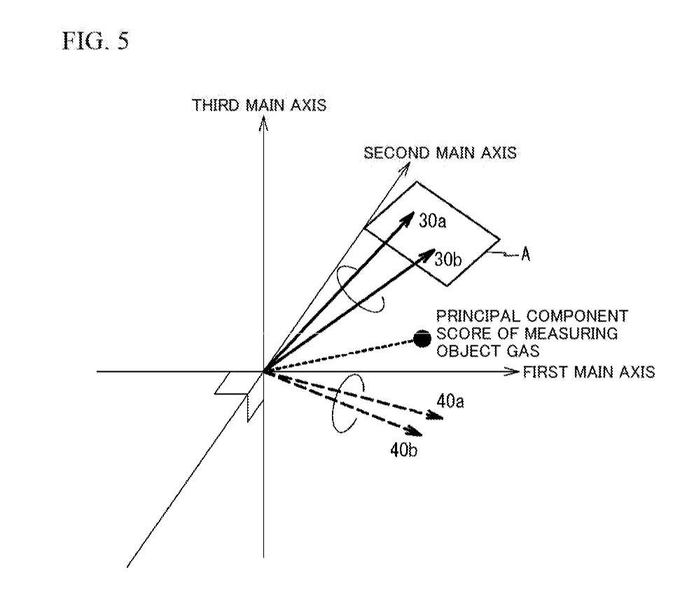

Here, it is preferable that the gas sensors 30a, 30b, 40a and 40b are configured so that the characteristic vectors of the gas sensors 30a and 30b are substantially orthogonal to the characteristic vectors of the gas sensors 40a and 40b. For example, this can be achieved by selecting principal materials of the gas sensitive members 34 and 43, as described above. When only the gas sensors 30a and 30b are used, only the responses of the oxide semiconductors are used. In this case, a difference between the characteristic vectors becomes small, and therefore an analysis space A becomes narrow, as illustrated in FIG. 5. On the contrary, in the present embodiment, the gas sensors 40a and 40b equipped with the gas sensitive members 43 having the characteristic vectors orthogonal to the characteristic vectors of the gas sensors 30a and 30b are used, so that a wide analysis space can be generated.

Next, the measuring object gas is introduced in the chamber 10 from the second inlet 12. The impedance measuring circuit 61 measures the responses (the impedance changes of a predetermined time) of the gas sensors 30a, 30b, 40a and 40b with respect to the measuring object gas, and stores the measurement result into the memory 63 as the matrix data. Next, the calculation circuit 62 performs the principal component analysis for the measurement result stored into the memory 63 to calculate a principal component score.

For example, in a space B which main axes constitute, the existence of the characteristic vectors (a11, a12, a13) of the gas sensor 30a, the characteristic vectors (b11, b12, b13) of the gas sensor 30b, the characteristic vectors (a21, a22, a23) of the gas sensor 40a, the characteristic vectors (b21, b22, b23) of the gas sensor 40b and the principal component score (PC1, PC2, PC3) of the measuring object gas can be confirmed as illustrated in FIG. 5. That is, in the space B, the vectors indicative of a characteristic of each gas sensor and the principal component score of the measuring object gas can be confirmed. In this case, the principal component score is a vector quantity. Therefore, it is difficult to determine what kind of component and how much concentration the principal component score is.

Consequently, the calculation circuit 62 multiplies the characteristic vector of the gas sensor 30a by the principal component score to calculate the gas concentration characterized by the gas sensor 30a. The calculation circuit 62 multiplies the characteristic vector of the gas sensor 30b by the principal component score to calculate the gas concentration characterized by the gas sensor 30b. The calculation circuit 62 multiplies the characteristic vector of the gas sensor 40a by the principal component score to calculate the gas concentration characterized by the gas sensor 40a. Moreover, the calculation circuit 62 multiplies the characteristic vector of the gas sensor 40b by the principal component score to calculate the gas concentration characterized by the gas sensor 40b.

Thus, a scalar quantity which is an index of the concentration is calculated with the use of the principal component score and the characteristic vector of the sensor. For example, an inner product of the characteristic vector and a coordinate of the principal component score is calculated as the scalar quantity. A concentration conversion value of the measuring object component of the gas sensor 30a is expressed by formula (1).

C1a11×PC1+a12×PC2+a13×PC3 (1)

A concentration conversion value of the measuring object component of the gas sensor 40a is expressed by formula (2).

C2=a21×PC1+a22×PC2+a23×PC3 (2)

In a statistical method such as the principal component analysis method, the axis is changed so as to express most data in the few axes. That is, a direction of the main axis is changed according to the population of data. For this reason, a difference due to the distribution of the component of the measuring subject gas can be expressed in a coordinate system of the main axes, but the expression does not express what kind of component is different and how much the quantity of the component is different.

However, in the present embodiment, the characteristic vector of the gas sensor 30a indicates a direction of the reducing gas, and the characteristic vector of the gas sensor 40a indicates a direction of the basic gas. Therefore, in the inner product of the characteristic vector and the principal component score, a reducing gas concentration C1 and a basic gas concentration C2 are stably acquired. That is, ammonia and the other reducing gases can be separated.

Each of the concentration conversion values acquired by the above-mentioned formulas (1) and (2) is a quantity depending on the concentration. In the present embodiment, to acquire more accurate concentration conversion values, two following processing is performed. One processing is unification of the units. The one processing is to unify the sets (z11, z12, . . . , z1n, and so on) of an input with the component having the concentration to be measured. The principal component score is calculated without standardization. The characteristic vector is made a unit vector.

The other processing is to make the above-mentioned formulas (1) and (2) into a square root of a square sum. For example, a following formula (3) is calculated as the concentration conversion value of the gas sensor 30a.

C1=√{(a11×PC1)²+(a12×PC2)²+(a13×PC3)²} (3)

Moreover, a following formula (4) is calculated as the concentration conversion value of the gas sensor 40a.

C2=√{(a21×PC1)²+(a22×PC2)²+(a23×PC3)²} (4)

The quantitativity can be acquired by the above-mentioned formulas (3) and (4). Here, it is assumed that the resistance change of the gas sensitive member linearly replies, for example, to the concentration of the gas in the same way. When a difference occurs in a response curve depending on the gas component or the concentration, the quantitativity might decrease. Even in such a case, a magnitude relation of the concentration conversion values is maintained. When the orthogonality of CuBr is confirmed beforehand, the quantitativity is acquired by using a value calculated from a sensor output of CuBr as ammonia concentration, and therefore the value calculated from the sensor output of CuBr may be used.

According to the present embodiment, each of the gas sensors 40a and 40b uses the gas sensitive member 43 mainly composed of the halide or the oxide of Cu or Ag. The gas sensitive member 43 has high sensitivity with respect to the basic gas, and has low sensitivity with respect to the reducing gas. Thereby, a high measurement accuracy with respect to the basic gas concentration is acquired. Next, each of the gas sensors 30a and 30b uses the oxide semiconductor mainly composed of at least one of Sn, W, Zn and In or the semiconductor mainly composed of C, as the gas sensitive member 34. The gas sensitive member 34 has high sensitivity with respect to the reducing gas. Thereby, a high measurement accuracy with respect to the reducing gas concentration is acquired. The gas sensitive member 34 has sensitivity with respect to the basic gas, but the basic gas concentration can be measured with the gas sensitive member 43, and therefore the sensitivity of the gas sensitive member 43 with respect to the basic gas can be excluded. Thus, an excellent selection ratio can be obtained with respect to the gas containing both of the reducing gas and the basic gas.

In the present embodiment, the concentration conversion value with respect to each gas sensitive member 34 of the gas sensors 30a and 30b is calculated, and the concentration conversion value with respect to each gas sensitive member 43 of the gas sensors 40a and 40b is calculated, but a calculation method of the concentration conversion value is not limited to this. For example, an arithmetic mean of the characteristic vectors of the gas sensitive members 34 of the gas sensors 30a and 30b may be used as a single characteristic vector, and an arithmetic mean of the characteristic vectors of the gas sensitive members 43 of the gas sensors 40a and 40b may be used as a single characteristic vector. In this case, an amount of calculation can be reduced. Moreover, the resistance changes of the gas sensitive members 34 and 43 may be corrected in accordance with at least one of the temperature and the humidity in the chamber 10 detected by the temperature and humidity sensor 50.

By the way, in the body of the animal including the human, in the case of the decomposition of a protein in a digestive organ, nitrogen occurs as ammonia. Alternatively, microbes and anaerobic bacteria living in the stomach and intestines decomposes urea with the use of a urease enzyme, thereby generating ammonia. A part of ammonia is absorbed in blood, and the remainder is exhausted outside the body as excrement. The blood including the nutrition absorbed from the digestive organ is collected as portal vein by a liver.

In the liver, the absorption of the nutrient matter is carried out, and metabolism having a detoxification function is carried out for toxin. Ammonia is the latter, is metabolized by a cycle called an intrahepatic urea cycle and is converted into urea. Then, the urea is filtered with kidney and is excreted with urine. When the human carries out vigorous exercise and muscle gets tired, ammonia occurs in the blood, and ammonia is metabolized by the similar urea cycle in the liver through a vein and is converted into the urea.

With such a metabolic function, the ammonia concentration in the living body is maintained to a constant level or less. Therefore, when the metabolic function of the liver has a disease and a liver function decreases, an ammonia concentration increases, and in a state of undernutrition, the ammonia concentration decreases. However, it may be said that a creature certainly contains ammonia in the blood as far as the creature has a nutrient and practices exercise. Since ammonia is vaporized by capillaries of the lungs and the skin, the breath and the sweat of the creature certainly would contain a very small amount of ammonia.

In a process that decomposes hydrocarbon, alcohols such as ethanol occur. Moreover, in the case of the decomposition of saccharide, ketones such as acetone occur. In the case of the decomposition of cholesterol, isoprene occurs. Moreover, in the diseases such as cancers, various VOCs occur due to the oxidation stress in a diseased part and are vaporized from the lungs and the skin via the blood.

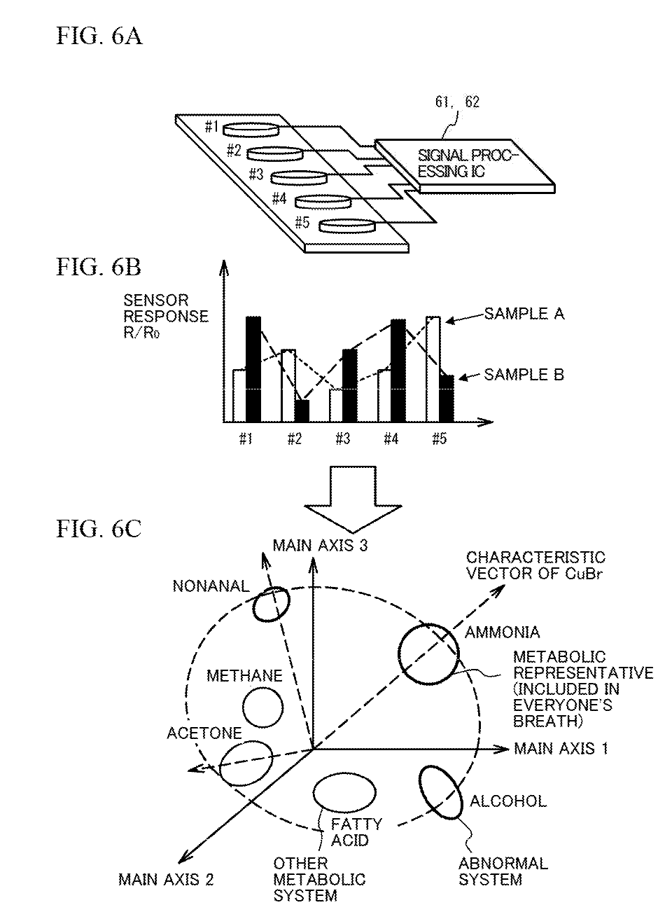

According to the present embodiment, ammonia can be separated from among various metabolic gases. The present embodiment is applied and a gas sensor having a third orthogonality is further provided, so that the number of detectable gas components increase and an electronic nose can be achieved. A plurality of gas sensors #1-#5 having respective different characteristics are provided as illustrated in FIGS. 6A and 6B and the response of each of the gas sensors is measured, so that the electronic nose called a breathprint sensor for comprehending the characteristics of components of the breath and the sweat can be configured as with a fingerprint. For example, the gas sensors 30a and 30b are used as the gas sensors #1 and #2, the gas sensors 40a and 40b are used as the gas sensors #3 and #4, the gas sensor having the third orthogonality is used as the gas sensor #5. Each of the gas sensors need to be connected to a signal processing IC corresponding to the impedance measuring circuit 61 and the calculation circuit 62 of FIG. 2. FIG. 6B illustrates a ratio of an initial resistance value and a resistance value after the gas introduction with respect to each gas sensitive member, as the response.

For example, the concentration of each gas component is calculated in the space B that the main axes constitute, as illustrated in FIG. 6C. For example, the relative coordinate of each gas component can be created on the basis of ammonia, a distribution of the direction and the strength of each gas component can be created, and characteristics of the distribution can be extracted with the use of pattern matching.

For example, ammonia (it is a metabolic representative and included in everyone's breath) corresponding to a central point, and each gas component as a characteristic point, e.g. nonanal which is suggested as the candidate of the biomarker of the lung cancer are measured. These components are separated by a means of the present embodiment and a pattern analysis of a concentration index is carried out, so that the change of the components in the breath by the lifestyle can be checked continuously without the pain such as the blood collection due to the simplicity of this technique. The breathprint sensor is mounted on a smart device and a wearable device, so that the devices can serve as a means that continues to analyze these gases with the simplicity such as a thermometer. This technique is made use of as a screening means for the improvement of the lifestyle and the early detection of the disease.

### EXAMPLE

An inventor performed an experiment with the use of the gas analyzer 100 of FIG. 2. CuBr was used as the gas sensitive members 43 of the gas sensors 40a and 40b. Coating was not applied to the gas sensitive member 43 of the gas sensor 40a. A Teflon-containing water-repellent coat was applied to the surface of the gas sensitive member 43 of the gas sensor 40b. Hereinafter, the measurement result of the gas sensor 40a is referred to as CuBr1, and the measurement result of the gas sensor 40b is referred to as CuBr2.

In the example, a gas sensor 30c was used in addition to the gas sensors 30a and 30b. The gas sensor 30c has a configuration illustrated in FIGS. 3A and 3B. The gas sensors 30a to 30c were arranged closer to the outlet 13 than the gas sensors 40a and 40b. A mixture of SnO (tin oxide) and WO₃ (tungsten oxide) was used as the gas sensitive members 34 of the gas sensors 30a to 30c. In each gas sensitive member 34, a compounding ratio and an oxidation degree of SnO and WO₃ are changed. Hereinafter, the measurement result of the gas sensor 30a is referred to as MOS (NH₃). The measurement result of the gas sensor 30b is referred to as MOS (RED). The measurement result of the gas sensor 30c is referred to as MOS (OX).

Five kinds of gases diluted by the atmosphere were introduced into the chamber 10, and the impedance change was measured in each sensor. Ammonia 1 ppm, acetone 10 ppm, ethanol 180 ppm, acetaldehyde 10 ppm and hydrogen sulfide 0.1 ppm were used as the five kinds of gases.

After the resistance of each sensor began to change, a ratio of a resistance value r after 10 seconds and a resistance value r0 after 0 seconds is taken. Since the resistance of the oxide semiconductor decreases due to the reducing gas, a formula “1−r/r0” is set as the change amount. Since the resistance of CuBr increases due to the basic gas, the resistance change rate was calculated with the use of a formula “r/r0−1”. In the principal component analysis, data is standardized.

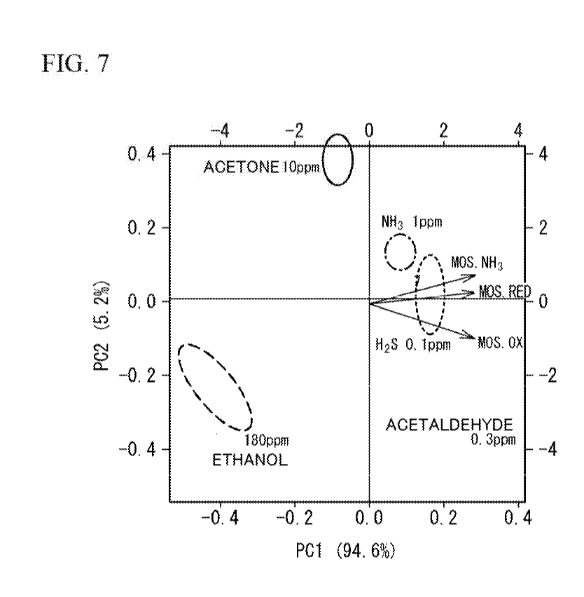

First, the principal component analysis was performed with the use of only the measurement result MOS (NH₃), the measurement result MOS (RED) and measurement result MOS (OX). FIG. 7 is a diagram illustrating a result of the principal component analysis. FIG. 7 corresponds to the above-mentioned analysis space A. In the example of FIG. 7, a contribution rate of the first main axis PC1 becomes 94.6%, and this suggests that most responses are represented by the PC1. Moreover, the characteristic vector of each sensor turns to the almost same direction, and hence there is no orthogonality. Since each sensor reacts to all reducing gases, this suggests that it is difficult to identify ammonia when the characteristic vector and the principal component change for each population.

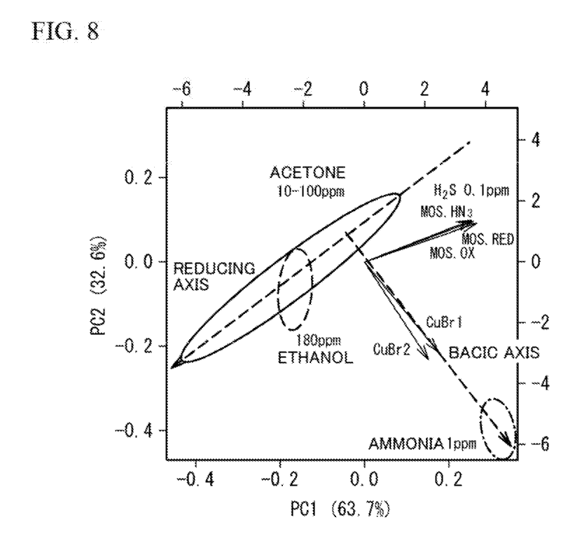

Next, similarly, the principal component analysis with respect to the five kinds of gases was performed with the use of a set including the measurement result CuBr1 and the measurement result CuBr2. FIG. 8 is a diagram illustrating a result of the principal component analysis. As illustrated in FIG. 8, this suggests that the contribution rate of the PC1 becomes 63.7%, the contribution rate of the PC2 becomes 32.6%, and 96.3% of data is shown in total.

It could be confirmed that the characteristic vectors of the oxide semiconductors (SnO₂ and WO₃) were substantially orthogonal to the characteristic vectors of the CuBr. Directions of the characteristic vectors of the CuBr indicate a basic. Therefore, in the characteristic vectors of the CuBr, only ammonia is arranged from among the five kinds of gases. A reason why the characteristic vectors have slight deviance with respect to the orthogonality is that the oxide semiconductors react to also ammonia and have the same component as the CuBr sensors.

On the other hand, it could be confirmed that the other reducing gases turn to a direction completely orthogonal to a basic axis. When data which changed a concentration of acetone between 10-100 ppm was added, a change on the reducibility could be confirmed in accordance with the concentration of acetone. It was difficult for this sensor characteristic to separate acetone and ethanol, but it was confirmed to be able to separate only ammonia. In addition, although there is only the contribution rate of around 3% of a remainder, not shown, acetone and ethanol are more likely to separate by using the third main axis PC3. This is approximately equal to the second main axis of a result (FIG. 7) of only MOS systems without CuBr. Ammonia becomes a remaining classification separated by the basic axis.

As described above, CuBr is added to the sensor arrays of the metal oxide, so that the separation of ammonia which was difficult conventionally is enabled, and a possibility that makes the separation of the remaining gas easy increases.

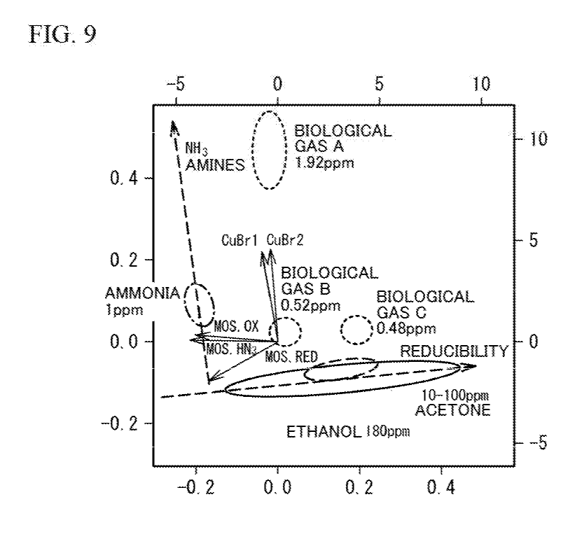

Therefore, real measurement results of the biological gases were added to the populations of the five kinds of experiment gases which were already generated. The biological gases were collected from three kinds of individuals. FIG. 9 is a diagram illustrating a result of the principal component analysis. In FIG. 9, the contribution rate of the PC1 becomes 46.7%, the contribution rate of the PC2 becomes 42.3%, and 89% of data is shown in total. From this result, it could be confirmed that, with respect to biological gases A and B, NH₃ concentrations differ from each other and the reducing gas components are the same level, and with respect to biological gases B and C, the NH₃ concentrations are the same level but the reducing gas component of the biological gas C is large.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various change, substitutions, and alterations could be made hereto without departing from the spirit and scope of the invention.

What is claimed is:

1. A gas analyzer comprising:
a chamber;
a first gas sensor provided in the chamber and including a first gas sensitive member;
a second gas sensor provided in the chamber and including a second gas sensitive member; and
a detector that detects each of resistance changes of the first and the second gas sensitive members;
wherein the first gas sensitive member is an oxide semiconductor mainly composed of at least one of Sn, W, Zn and In or a semiconductor mainly composed of C, and
the second gas sensitive member is mainly composed of a halide or an oxide of Cu or Ag.

2. The gas analyzer as claimed in claim 1, wherein
the detector multiplies a characteristic vector of a response of the first gas sensitive member with respect to a gas by a matrix including the resistance change of the first gas sensitive member, and detects a result of multiplication as a concentration of a reducing gas.

3. The gas analyzer as claimed in claim 2, wherein
the characteristic vector of the response of the first gas sensitive member is acquired by performing a statistics processing or a machine learning on a resistance change rate of the first gas sensitive member to a gas having a well-known component and a well-known concentration.

4. The gas analyzer as claimed in claim 1, wherein
the detector multiplies a characteristic vector of a response of the second gas sensitive member with respect to a gas by a matrix including the resistance change of the second gas sensitive member, and detects a result of multiplication as a concentration of a basic gas.

5. The gas analyzer as claimed in claim 4, wherein
the characteristic vector of the response of the second gas sensitive member is acquired by performing a statistics processing or a machine learning on a resistance change rate of the second gas sensitive member to a gas having a well-known component and a well-known concentration.

6. The gas analyzer as claimed in claim 1, wherein
the first gas sensor includes a heater for heating the first gas sensitive member, and
the second gas sensor is arranged at an upstream side in a flowing direction of a gas in the chamber than the first gas sensor.

7. A gas analysis method implemented by a gas analyzer provided with a first gas sensor including a first gas sensitive member and a second gas sensor including a second gas sensitive member, the gas analysis method comprising:
detecting each of resistance changes of the first and the second gas sensitive members;
wherein the first gas sensitive member is an oxide semiconductor mainly composed of at least one of Sn, W, Zn and In or a semiconductor mainly composed of C, and
the second gas sensitive member is mainly composed of a halide or an oxide of Cu or Ag.

8. The gas analysis method as claimed in claim 7, wherein
the detecting multiplies a characteristic vector of a response of the first gas sensitive member with respect to a gas by a matrix including the resistance change of the first gas sensitive member, and detects a result of multiplication as a concentration of a reducing gas.

9. The gas analysis method as claimed in claim 8, wherein
the characteristic vector of the response of the first gas sensitive member is acquired by performing a statistics processing or a machine learning on a resistance change rate of the first gas sensitive member to a gas having a well-known component and a well-known concentration.

10. The gas analysis method as claimed in claim 7, wherein
the detecting multiplies a characteristic vector of a response of the second gas sensitive member with respect to a gas by a matrix including the resistance change of the second gas sensitive member, and detects a result of multiplication as a concentration of a basic gas.

11. The gas analysis method as claimed in claim 10, wherein
the characteristic vector of the response of the second gas sensitive member is acquired by performing a statistics processing or a machine learning on a resistance change rate of the second gas sensitive member to a gas having a well-known component and a well-known concentration.
